(19) United States
(12) Patent Application Publication
(10) Pub. No.: US 2009/0259106 A1
(43) Pub. Date: Oct. 15, 2009

(54) RETRACTOR SYSTEM FOR SURGICAL APPLICATIONS FOR DETECTING CHARACTERISTIC PARAMETERS OF ORGANIC TISSUES

(76) Inventors: Giuseppe Catapano, Benevento (IT); Sabato Santaniello, Nola (Napoli) (IT); Luigi Glielmo, Napoli (IT); Giovanni Fiengo, Ercolano (Napoli) (IT)

Correspondence Address: ALLEN, DYER, DOPPELT, MILBRATH & GILCHRIST P.A., 1401 CITRUS CENTER 255 SOUTH ORANGE AVENUE, P.O. BOX 3791, ORLANDO, FL, 32802-3791, US

(21) Appl. No.: 12/281,185

(22) PCT Filed: Mar. 2, 2007
(86) PCT No.: PCT/IT2007/000155

§ 371 Date: Dec. 1, 2008

(30) Foreign Application Priority Data
Mar. 2, 2006 (IT) NA2006A000024

(51) Int. Cl. A61B 1/32

(52) U.S. Cl. 600/202

(57) ABSTRACT

This patent describes a retractor system for surgical applications of the type used to retract organic tissues not involved in an operation, in an area where such an operation is to be performed, of the type comprising at least one spatula equipped with at least one spatula body (1) and a layer of bio-compatible material (20), as a coating adhering to the spatula body (1). Advantageously according to the invention, the retractor system comprises at least one sensor (2, 3, 4, 27), substantially integrated in such a spatula body (1) and at least partially coated with the layer of bio-compatible material (20) to detect characteristic parameters of the organic tissues at a point of contact with the spatula body (1). A detection system (40) using such retractor system is also described.

### FIELD OF APPLICATION

The present invention refers to a retractor system for surgical applications for detecting characteristic parameters of organic tissues.

More specifically, the invention refers to a retractor system for surgical applications of the type used to retract, in an area involved in an operation, organic tissues not involved in such an operation, of the type comprising at least one spatula equipped with a spatula body and a layer of bio-compatible material, as a coating adhering to said spatula body.

The invention also refers to a detection system using such a retractor system.

### PRIOR ART

As is well known, to carry out a surgical intervention sometimes the operator or surgeon has to free the operated area from possible organic tissues not involved in the operation.

The procedure typically used for this purpose foresees that the operator exploits slits already existing in the tissues, or he creates new ones, then pulling them apart by means of a retractor system like for example sterile steel or silicon spatulas. Once the created cavity is the right size to be able to perform the actual intervention, the position of the spatulas is locked by means of suitable articulated mechanical support systems (for example, so-called “Leyla retractors”).

The main problem of this sort of practice is the absolute impossibility of determining the pressure actually exerted by the spatula on the tissue with which it is in contact. Moreover, this pressure represents one of the parameters of greatest risk in a vast range of interventions. For example, in neurosurgery, an excessive pressure exerted on specific portions of the brain can cause serious damage to the patient's brain activity, with possible consequences on the voluntary and involuntary functions.

One of the reasons why such an excessive pressure exerted by the spatula involves risk is, for example in the case considered here, the effect that it has upon the oxygenation of the tissues with which it is in contact: a pressure exerted from the outside, indeed, temporarily modifies the morphology of such a tissue. If such a modification is excessive it has the effect of altering the saturation level of oxygen (SO2) in the region of the tissues along the contact surface with the spatula, with a consequent reduction of the electrical activity and possible necrosis of the nerve cells located here.

It is also known that a direct measurement of the oxygen saturation level (also known as “oximetry”) in the tissues is only currently partially possible and not for all areas of the human body.

The oximetry sensor devices currently developed indeed allow either a measurement of the peripheral arterial oximetry or a measurement of the capillary oximetry (both arterial and venous) of the brain. Such devices have the advantage of being non-invasive, since they carry out the measurement exploiting infrared spectroscopic technologies, with relatively fast response (real time measurements every 5 seconds) but with low spatial resolution and a limited field of use. In particular, sensor devices that provide measurement of the peripheral oximetry, for example, can only be applied to the extremities of the patient (toes and/or fingers), thus not being very useful for evaluating risk situations like those outlined previously. Sensor devices for capillary oximetry, on the other hand, can currently be applied to the patient's frontal sinuses and are limited to providing an average measurement for the monitored region, which is about half the size of the forehead.

Finally, it should be emphasised that in the current state of the art, the range of sensor devices for measuring oximetry that have been developed do not allow either measurements (localised on regions having a diameter of a few millimetres) of capillary oximetry or combined measurements of this and the resulting external pressure.

In particular, in the field of neurosurgery, the combined ability to know pressure and oximetry measurements is of particular interest since, together with the evaluation of the electrical neurone activity level, it allows the maximum pressure, in relation to the type and duration of the intervention, that can be exerted with a surgical spatula on the brain tissue without the consequent reduction of the oximetry producing necrosis of neurones with damage to the patient, to be established.

Although such damage linked to the pressure of the spatula on the brain tissue has been widely reported in literature, to date no aid has been developed that allows such risks from prolonged use of a spatula on the brain to be reduced.

The technical problem at the basis of the present invention is that of devising a retractor system suitable for detecting characteristic parameters of at least one portion of tissue involved in the retracting operation and having structural and functional characteristics such as to overcome the limitations and drawbacks that still afflict sensor devices made according to the prior art.

### SUMMARY OF THE INVENTION

The solution idea at the basis of the present invention is to integrate at least one sensor of such characteristic parameters into the body of a retracting spatula of the retractor system.

Based upon such an idea of solution the technical problem is solved by a retractor system of the type indicated previously and defined by the characterising part of claim 1.

The problem is also solved by a detector system of the type indicated previously and defined by the characterising part of claim 28.

The characteristics and advantages of the retractor system and of the relative detector system according to the invention shall become clearer from the following description of an example embodiment thereof, given for indicating and not limiting purposes with reference to the attached drawings.

### BRIEF DESCRIPTION OF THE DRAWINGS

In such drawings:

FIG. 1 schematically shows a system for detecting characteristic parameters of organic tissues according to the invention;

FIG. 2 schematically shows a front view of a portion of surgical spatula of the retractor system according to the invention;

FIG. 3 schematically shows an axonometric view of a portion of surgical spatula of the retractor system according to the invention;

FIGS. 4 and 5 schematically show section views of a portion of surgical spatula of the retractor system according to the invention;

FIGS. 6 and 7 schematically show front views of a portion of surgical spatula of the retractor system according to the invention;

FIGS. 8 and 9 schematically show axonometric partial section views of a portion of surgical spatula of the retractor system according to the invention;

FIG. 10 schematically shows a section view of a portion of surgical spatula of the retractor system according to the invention;

FIG. 11 schematically shows a front view of a portion of surgical spatula of the retractor system according to the invention;

FIG. 12 schematically shows front views of variant embodiments of a portion of surgical spatula of the retractor system according to the invention;

FIG. 13 schematically shows a front view of a portion of surgical spatula of the retractor system according to the invention;

FIG. 14 schematically shows an axonometric partial section view of a portion of surgical spatula of the retractor system according to the invention;

FIGS. 15 and 16 schematically show axonometric views of a detail of the surgical spatula of the retractor system according to the invention; and

FIGS. 17 and 18 schematically show axonometric partial section views of a portion of surgical spatula of the retractor system according to the invention.

### DETAILED DESCRIPTION

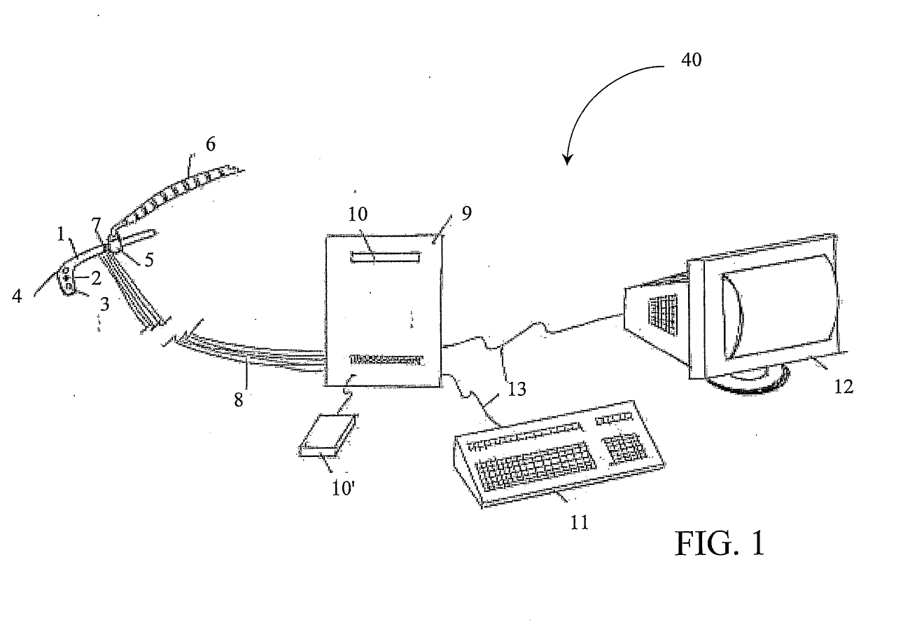

With reference to such figures, and in particular to FIG. 1, hereafter a system for detecting characteristic parameters of organic tissues according to the invention is described, wholly indicated with 40.

Such a detection system 40 essentially comprises a spatula 1 for surgical applications that has a base surface that, during the course of an operation, goes into contact with organic tissues, in particular the tissues of the patient being operated upon.

Advantageously, according to the invention and as shall be made clearer in the rest of the description, the spatula 50 comprises at least one sensor of characteristic parameters of such organic tissues.

In the example illustrated in FIG. 1, the spatula 50 comprises, in particular, inside its spatula body 1, an infrared oximetry sensor 2, a force sensor 3 and an electrode 4 for registering the electrical neurone activity.

The spatula 50 is also connected, through an articulated joint 5 with a locking screw, to an end arm of a suspension system 6 of the “Leyla's retractor” type.

Inside the joint 5, at the locking screw, a further force sensor is inserted to evaluate the force exerted by the screw to lock the spatula 50.

The sensors are also connected, through electric cables 8 coated with an insulating sheath that can be sterilised, to a microprocessor calculation system 9, inside which at least one electronic measuring system is integrated. The unit of the microprocessor calculation system 9 and the electronic measuring system acquires measurement signals generated by such sensors, processes them suitably and, finally, stores their information content in suitable storage supports, like for example optical mass supports through a fixed writer 10 or else of the plug & play type 10′.

The microprocessor calculation system 9 interfaces with the user through a keyboard 11, for receiving commands from the user, and a monitor 12, for displaying the signals currently acquired and processed and control messages generated by suitable calculation routines executed by the microprocessor calculation system 9 itself. The connection between the microprocessor calculation system 9, the keyboard 11 and the monitor 12 takes place through electrical wires 13 coated with an insulating sheath that can be sterilised.

In particular, the proposed detection system has a high spatial resolution, high flexibility and the ability to make and correlate force, oximetry and, possibly, electrical neurone activity measurements.

Moreover, in order to increase the amount of information that the surgeon can use for the clinical intra-operatory evaluation of the patient, it is advantageously foreseen to equip the spatula body 1 with a further sensor capable of also monitoring the temperature of the portion of organic tissue exposed to contact with the spatula.

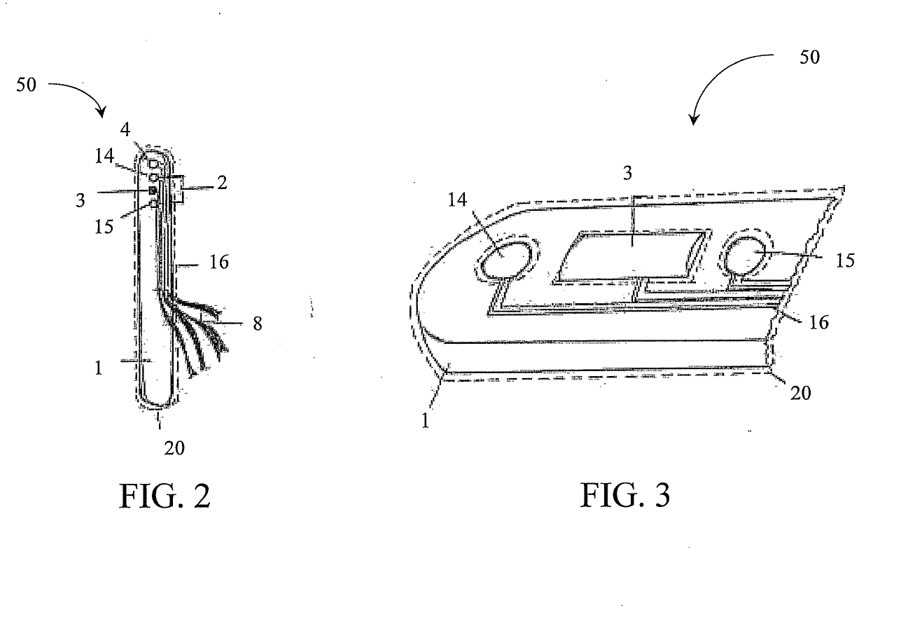

The present invention thus refers to a retractor system of the type comprising at least one spatula for surgical uses, hereafter indicated simply as spatula 1 and illustrated in FIGS. 2-18.

In particular, the body 1 of the spatula 50 is suitably modified to receive, at a work surface S, intended to come into contact with organic tissues, at least one sensor of characteristic parameters of such organic tissues. In the example illustrated in FIG. 3, the spatula body 1 comprises a pair of small sensors (for example, made through MEMS—Micro Electro Mechanical Systems—technology. In particular, the spatula body 1 comprises a force sensor 3, for example made through a load cell or else an extensometer, for evaluating the force exerted locally by the spatula body 1 on the tissue touched, as well as a sensor 2 for measuring capillary oximetry, for example made with infrared spectroscopy technology and comprising at least one emitter 14 and a receiver 15.

In the case in which it is intended for neurosurgery interventions, the spatula 1 also comprises, again in its structure, an electrode 4, in particular a metal electrode for registering the electrical neurone activity of the portion of nerve tissue with which the work surface S of the spatula body 1 comes into contact, as illustrated in FIG. 2.

In particular, the electrical signals detected by such sensors 2, 3 and 4 are propagated towards a distal portion of the spatula 1 far from the work area (in other words from the point of contact of the spatula body 1 with the patient's organic tissues) by means of pathways 16 of conductive material integrated inside the spatula body 1. Said pathways 16 are connected, at the ends not in contact with the sensors, to electric wires 8 coated with an insulating sheath. Said sheath is intended to be sterile or able to be sterilised according to the procedures foreseen by the current sanitary standards of the national territory with regard to hygiene of surgical tools and apparatuses.

The unit of the spatula body 1 (with the exception of the areas occupied by the aforementioned sensors), of the pathways 16 of conductive material and of the junctions between them and the electric wires 8 is coated with a sterile and bio-compatible synthetic material, like for example silicon.

FIG. 2 therefore illustrates a portion of the detection system 40 according to the invention, in particular a portion of a spatula for surgical applications 50 in the spatula body 1 of which a force measuring sensor 3, an oximetry sensor 2 and an electrode 4 are integrated. Such sensors are advantageously arranged along the work surface S of the spatula 50 that comes into contact with the organic tissues of the subject undergoing the operation.

In particular, the oximetry sensor 2 of the example of the figure is developed with infrared spectroscopy technology and comprises an infrared ray emitter 14 and receiver 15.

The electrode 4 is used to register neurone activity, in particular for use of the spatula 50 in neurosurgery interventions.

It is worth noting the fact that the electrical signals detected by each of the aforementioned sensors are transmitted by means of conductive pathways 16 made along the work surface S of the spatula 50 and welded to electrical wires 8 coated with an insulating sheath that can be sterilised.

Moreover, FIG. 3 depicts a detail of the end (in particular the portion provided with sensors) of a possible embodiment of the surgical spatula 50 intended to come into contact with organic tissues at the site of the surgical intervention and equipped, along its work surface S, with the force sensor 3 and the oximetry sensor made up of the emitter 14 and the receiver 15 of infrared rays.

Advantageously, according to the invention, the spatula body 1 is configured so that optical and electronic components constituting the sensors are arranged inside it. In particular, they are arranged so that the emitter 14 and the receiver 15 have a profile that does not project with respect to a plane of the work surface S of the spatula 50. The pathways of conductive material 16 are also arranged along such a surface to connect the sensors to necessary power sources and to pass the signals to the acquisition and storage device.

Also advantageously, the spatula 50 and all of the optical and electronic components of the sensors 2, 3 and 4 are coated with a layer 20 of insulating and bio-compatible protective material. Exceptions to such coating are the emission surface of the emitter 14, the reception surface of the receiver 15 and the portion of the force sensor 3 intended to deform in contact with the organic tissues; as indicated with a broken line in FIG. 3.

From such a FIG. 3 it can also be seen that the size and the distances apart of the integrated sensors on the spatula 50 must be such that, at the moment when the contact is made between the spatula 50 and the organic tissue, the sensors can lie down on the latter, providing the desired measurements.

It should be noted that, given the characteristics of physiological and structural homogeneity of the organic tissue close to the spatula 50, the measurements obtained by the sensors are such as to be considered to be correlated.

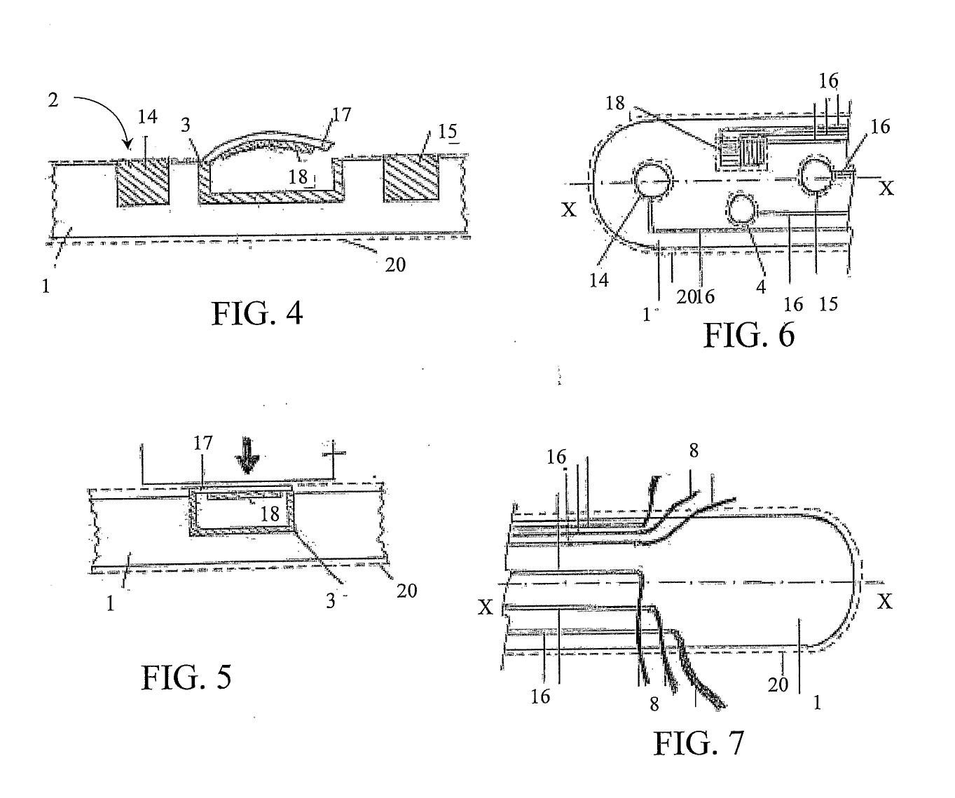

FIG. 4 represents a detail of the longitudinal section of the surgical spatula 50 in which the force sensor 3 and the oximetry sensor 2 are made. In particular, the force sensor 3 comprises a spring 17, elastically deformable under the action of a load to be measured, the resting profile of which can possibly exceed the profile of the work surface S of the spatula 50. An extensometer 18 is associated with such a spring 17, in particular welded to it.

Advantageously, the spring 17 is made from bio-compatible material and has sufficient flexibility to be able to deform in contact with organic tissues whenever the spatula 50 exerts pressure on them.

Moreover, the oximetry sensor 2 is made, like in the example illustrated in FIG. 3, with spectroscopy technology and comprises the infrared ray emitter 14 and, correspondingly, at least one receiver 15.

Like before, the emitter 14 and the receiver 15 are arranged so as not to project with respect to the profile of the spatula 50 along the work surface S.

The spatula 50, with the exception of the areas corresponding to the surfaces of the emitter 14, of the receiver 15 and of the spring 17, is always covered by the layer of insulating, sterile and bio-compatible material.

Advantageously, according to the invention, as illustrated in such a FIG. 4, the emitter 14 and the receiver 15 of the sensor for measuring the oximetry 2 have a profile that does not project with respect to the work surface S of the spatula 50. The electronic components of such an oximetry sensor, in particular for generating the probe signals used for measuring oximetry are, on the other hand, arranged inside the spatula body 1.

It is worth noting that, by using as force sensitive element an extensometer 18 welded to the spring 17 like in the example of FIGS. 4 and 5, the measurement provided by the sensor 3 depends upon the state of deformation of the spring 17. Therefore, such a spring 17, which must be made from bio-compatible material, must have sufficient flexibility to be able to deform in contact with organic tissues whenever the spatula exerts pressure on them. FIG. 5 shows as an example how the spring 17 is advantageously made in such a way that the possible deformations mainly have effects on its profile, which, in maximum load conditions, shall align with the outer surface, in other words with the work surface S of the spatula 50. For safety reasons, it is presumed in any case that, at rest, the height of the profile of the spring 17 with respect to the work surface S is less than the linear measurement of 1 millimetre.

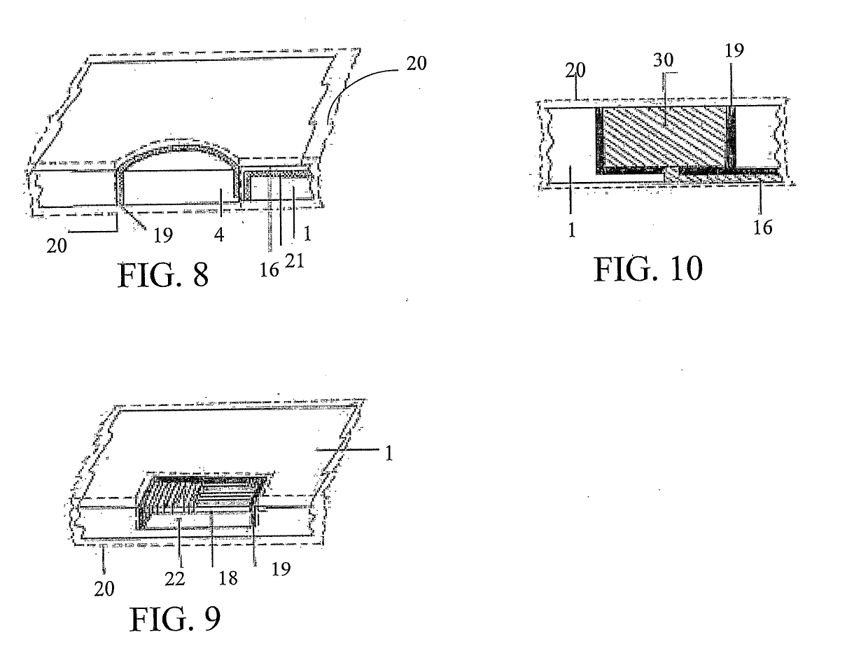

FIGS. 6 and 7 represent, in plan, two ends of a further embodiment, intended for neurosurgical applications, of the retractor system according to the invention. In this case the spatula body 1 is suitably configured so as to receive extensometers 18, with force sensor function, and electrode 4 for registering the electrical neurone activity and an oximetry sensor made with spectroscopy technology and comprising an emitter 14 and a receiver 15 of infrared rays. The extensometers 18 and the electrode 4 are aligned transversally to a longitudinal axis XX of the spatula body 1; the emitter 14 and the receiver 15 of the oximetry sensor, on the other hand, lie along such an axis XX. The arrangement of the aforementioned sensors is suitable so as to minimise the bulk inside the spatula body 1 (as also illustrated in FIGS. 8 and 9 described hereafter).

Grooves are made along the work surface S of the spatula body 1 to make pathways 16 of conductive material that, without emerging with respect to the profile of the spatula 50, allow the connection of the sensors with electrical power sources and an acquisition system of the signals generated. Said pathways 16 end near to a tip of the spatula 50 opposite the one where the sensors are positions, and here are connected to electric wires 8 coated with an insulated sheath that is sterile or able to be sterilised. Such wires 8 in turn connect with the power sources and the acquisition system, should these be arranged away from the site of use of the spatula 50, in this way making the detection system 40 according to the invention.

The pathways and the components of the sensors not in contact with the organic tissues are suitably coated with a film of sterile, insulating and bio-compatible material, in particular the layer 20.

FIG. 8 also shows a detail of the longitudinal section of the spatula body 1 suitably perforated to receive the electrode 4 for registering neurological signals. In the example embodiment illustrated in the figure, the electrode 4 is glued to the spatula body 1 by means of insulating adhesive material so as to be electrically disconnected from the spatula body 1 itself.

The electrode 4 is connected to a pathway of conductive material 16 to propagate the electrical signal acquired at the brain tissue towards the data acquisition system. Said pathway 16 extends along the work surface of the spatula 50 on a bed of insulating material 21 used to achieve the electrical decoupling between the pathway itself and the spatula 50. Both are coated with a film of sterile, insulating and bio-compatible material.

Also in this case, an outer layer 20 of sterile, insulating and bio-compatible material is foreseen that prevents the formation of ridges along both of the outer surfaces of the spatula 50.

FIG. 9 shows a different detail in longitudinal section of the spatula body 1 comprising extensometers 18, made from resistive material (for example aluminium or stainless steel) or piezoresistive material and arranged to form a single membrane, located in a cavity 22 suitably made inside the spatula body 1 and fixedly connected to the latter. In particular, said membrane is suspended above the cavity 22, and hinged to the ends by means of a layer of insulating adhesive material 19 such as to achieve the electrical decoupling between the extensometers 18 and the spatula 50. Such a membrane is positioned so that an interstice cavity remains between it and the spatula body 1, in which there can possibly be air at ambient pressure. The work surface S of the spatula 50 and the cavity 22 are not, however, in fluid communication with each other.

Suitably, the membrane is arranged so as to deform due to the application of pressures from the outside, thus permitting the detection by the extensometers 18. The spatula 50, with the exception of the work surface occupied by the extensometers 18, is coated with a layer 20 of sterile, insulating and bio-compatible material.

It should be noted that the hinging of such a membrane is such that if the membrane is subjected to a pressure through contact with organic tissues it deforms but does not move from the site in which it is arranged. The pressure measurement detected by the extensometers 18 is thus proportional to the deformation undergone by them.

It should also be noted that the interstice between the spatula body 1 and the surface of the membrane can be occupied by air at ambient pressure but, for reasons of hygiene and safety of the patient, it does not come into contact with organic tissues.

In a further embodiment of the system object of the present patent the pathways of conductive material needed to connect some or all of the sensors arranged on the spatula to the power sources and/or to the acquisition system can be made on a surface, hereafter indicated as base surface of the spatula 50, opposite the work surface S.

Yet another different embodiment foresees the use, as pressure sensor, of a load cell welded inside the spatula body 1 and arranged so that its useful measurement surface is aligned with the work surface of the spatula 50, as illustrated in FIG. 10.

In particular, the load cell 30 is arranged in a cavity 22 suitably made inside the spatula body 1. In order to achieve the electrical insulation between the spatula body 1, the load cell 30 and the pathway of conductive material 16 used to connect the cell to electrical power sources, a layer 19 of insulating adhesive material is introduced into the interstices.

Also in this case, the spatula 50, with the exception of the portion of surface corresponding to the load cell 30, and the pathway of conductive material 16 are coated with a layer 20 of sterile, insulating and bio-compatible material.

The thickness of the load cell 30 is advantageously limited so as not to exceed an external profile of the spatula 50 by more than 1 millimetre, in particular on the base surface opposite the work surface.

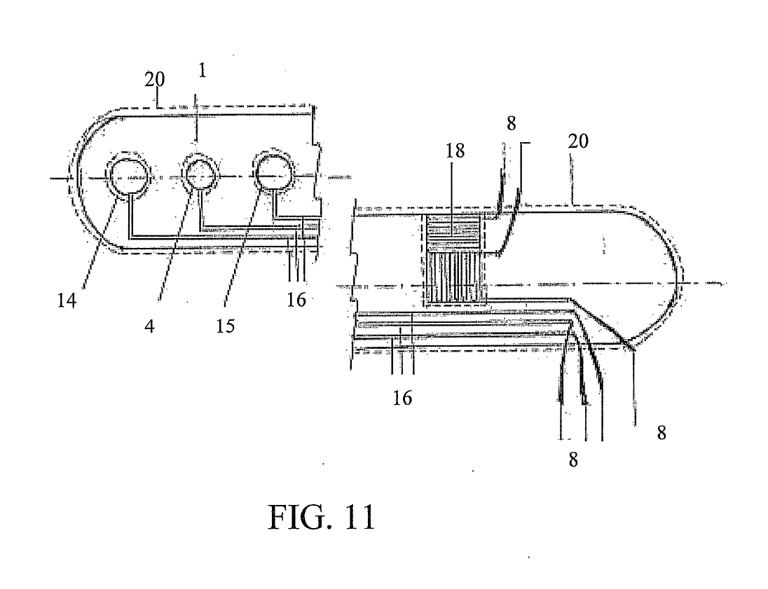

Another possible embodiment, on the other hand, foresees the use, as pressure sensor, of one or more extensometers 18 glued onto the work surface of the spatula 50 at an area not in contact with the organic tissues but adjacent to them, as illustrated for example in FIG. 11. Indeed, since an extensometer supplies a force measurement that is correlated to the degree of deformation that the force makes it undergo, it is assumed that the extensometer is arranged in the area of the surface of the spatula 50 where the deformation due to the pressure exerted by the organic tissue is greatest.

The extensometer is also coated with a layer 20 of silicon or else another sterile, insulating and bio-compatible material.

The oximetry sensor 2 and the electrode 4 for registering the electrical neurone activity, on the other hand, are suitably arranged, like in the case of the previous examples, so as to evaluate, respectively, the level of oxygenation of the tissues and the state of activity of the neurones at the area in contact with the spatula 50.

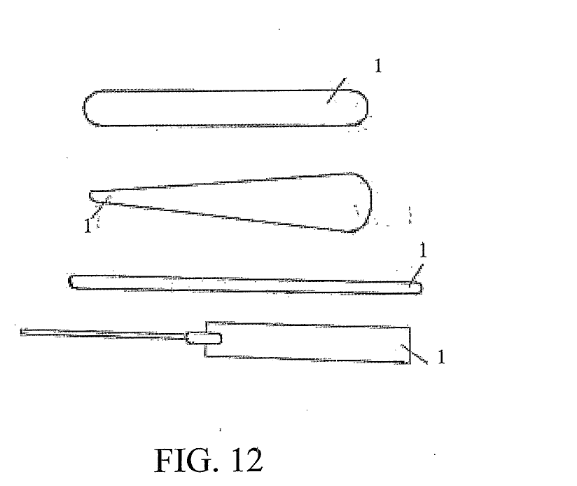

Examples of metal spatulas for surgical applications that are different to one another in shape and thickness of the portion intended to come into contact with organic tissues during a surgical intervention are schematically illustrated in FIG. 12.

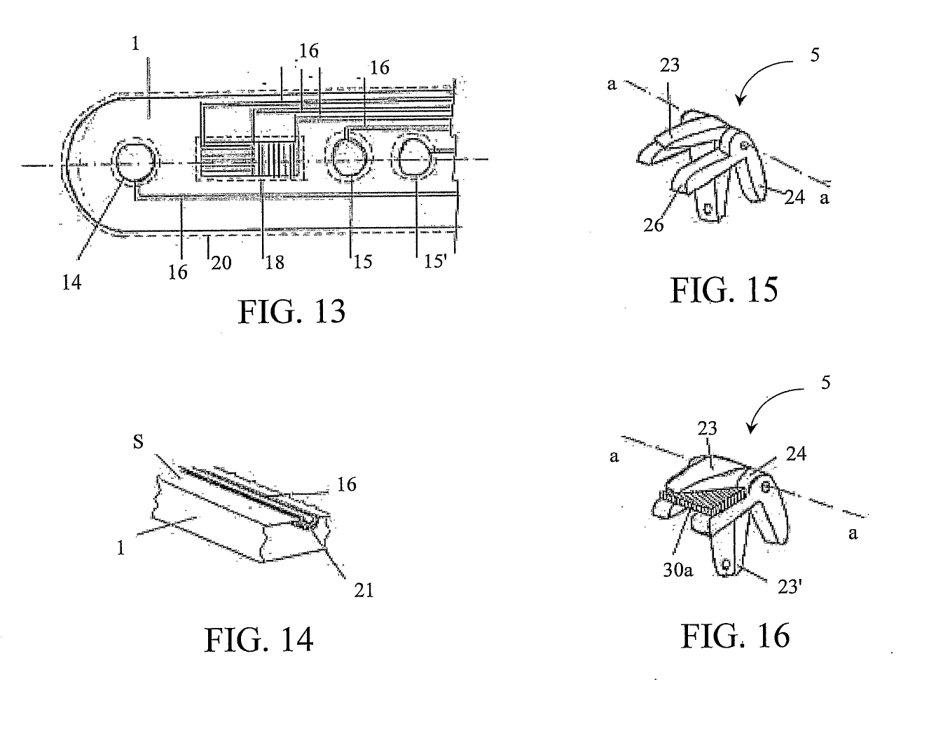

A further example embodiment of the spatula 50 of the retractor system 40 according to the invention is schematically illustrated in FIG. 13 and comprises a combination of extensometers 18 and an oximetry sensor made up of an emitter 14 and two receivers 15, 15′ of infrared rays. The connection between the aforementioned sensors and the electrical power sources or rather an acquisition system of the signals generated by them is achieved through pathways 16 of conductive material made along the work surface S of the spatula body 1.

Suitably, the pathways 16 of conductive material, so that they can propagate electrical signals without the risk of dispersion along the spatula, are housed on a layer 21 of insulating material laid out inside the spatula body 1 along the planned route for such pathways 16, as illustrated in FIG. 14.

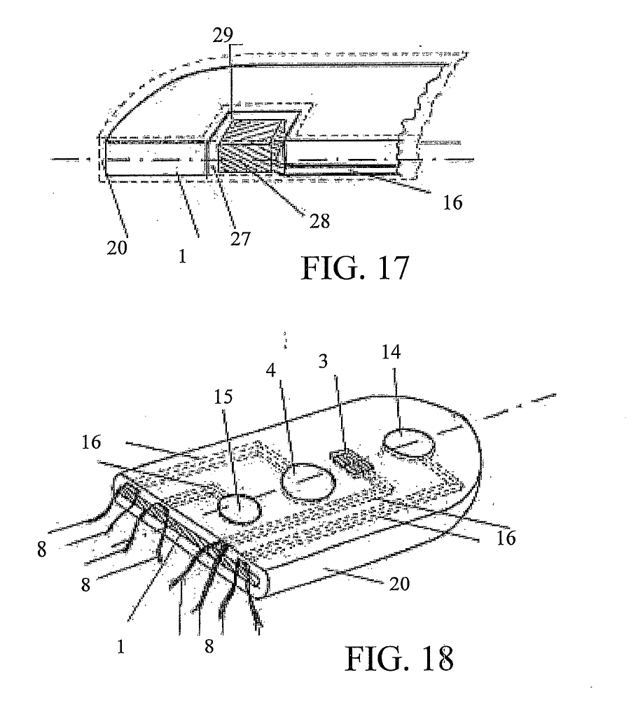

In a variant embodiment of the detection system 40 according to the invention, the spatula 50 also comprises, integrated in its spatula body 1, a temperature sensor 27, as illustrated in FIG. 17.

In particular, the temperature sensor 27 considered comprises a thermistor the linear dimensions of which are selected so as to be able to be arranged inside the spatula body 1. The connections with the electrical power sources and with an acquisition system take place, like in the example embodiments shown above, through pathways 16 of conductive material and/or electrical wires coated with an insulating sheath that is sterile or able to be sterilised.

The sensitive element of such a temperature sensor 27 is coated with a layer of bio-compatible material 29 that ensures that the device is sterile and electrical but not thermal insulation.

Also in this case, the temperature sensor 27 is sized so as not to project with respect to the work surface S of the spatula 50. Along the surface of the latter pathways of conductive material 16 are made to connect the sensor to the necessary power sources and to propagate the signals to suitable acquisition and storage systems.

As before, the spatula body 1 with the exception of the sensitive element of such a temperature sensor 27 is coated with a layer 20 of sterile, insulating and bio-compatible material.

It is also possible to use a surgical spatula 50 equipped with a sheath of sterile and bio-compatible coating material and integrating inside of it at least one of the pressure, oximetry, electrical activity registering and temperature sensors described previously and the conductive pathways 16 for the connection with such sensors, as illustrated in FIG. 18.

The arrangement of such sensors and the technology to be used are totally similar to those outlined in the examples shown above. In particular, the sensitive components must be in contact with the portion of organic tissue interacting with the coated spatula without, however, having a profile emerging from that of the outer surface of the sheath. Moreover, the connection between the circuit components of the sensors and the possible electrical power sources and/or the measurer must take place by means of electrical wires coated with an insulating sheath that is sterile or able to be sterilised according to the procedures foreseen by the current sanitary standards of the national territory with regard to hygiene of surgical tools and apparatuses.

As shown in FIG. 18, the sheath elastically adheres to the spatula 50, in particular to the spatula body 1, and deforms as a unit with it. The inside of the sheath is suitably shaped so as to house the circuitry of the various integrated sensors and the relative interconnections with the power sources and/or the measurer, whereas, along the surface of the sheath in contact with the organic tissue, openings are made in which the sensitive surfaces of the integrated sensors are arranged.

In this case, it is possible to suitably size the coating sheath to adapt it to surgical spatulas that already exist, thus making them sensorised for multi-parameter monitoring.

It should be noted that, from the functional point of view, the coating of a surgical spatula through a sheath layer sensorised according to the ways outlined above is totally equivalent to the integration of sensors inside the spatula body: the adherence of the sheath to the spatula and the arrangement of the sensors are such as to ensure that the values of the detected signals are practically equal to those that can be detected by integrating the same sensors inside the spatula body, just as was done in the case of the examples shown in FIGS. 2 to 11 and in FIGS. 13, 14 and 17.

In an alternative embodiment of the invention, at least one and preferably all of the sensors illustrated above are fibre optic sensors.

A further embodiment of the invention foresees that in at least one of the mechanical supports for keeping the spatula 50 in a fixed position in space a connection joint with the spatula 50 is foreseen, made through a terminal with locking screw, as illustrated in FIG. 15, inside of which a force sensor, in particular a load cell, is suitably inserted.

In its most general form, the joint 5 for connection to a suspension system 6 of the spatula, at a point of the spatula body 1 not coinciding with the point of contact with the organic tissues, is advantageously articulated and suitable for keeping the spatula body 1 in a predetermined position in three-dimensional space.

In particular, such a joint 5 comprises a rigid connection structure 24 with the spatula body 1 equipped with a projection 26 for housing a force measurer, in particular a load cell 30a, and associated with a mobile element 23 able to be moved in abutment onto the force measurer 30a to hold it.

In the example of FIG. 16, it can be seen how the load cell 30a, welded to a rigid support structure of the joint 5 or terminal at one of its two arms, has the task of supplying the measurement of the pressure exerted by the arm on the spatula inserted in the terminal. In the theoretical case in which the spatula 50 is rigidly attached to the terminal by means of the locking screw, said measurement is correlated to the force exerted by the spatula on the tissue with which it is in contact. Therefore, it can be used as additional information for identifying the nature of the pressure exerted by the spatula on the tissue, supplying an indirect estimation thereof, in the case in which the sensor arranged on the surface of the spatula breaks, and making it possible to establish how said pressure discharges onto the mechanical support structure.

In particular, the articulated joint of FIGS. 15 and 16 can be used for the interconnection between a surgical spatula and a suspension system of the “Leyla's retractor” type. In particular, the joint 5 comprises a locking screw 23 and a rigid support structure or “terminal” 24: the former is mobile about its rotation axis aa and can be locked through stops whereas the latter is usually firmly connected to the suspension system. The use of the joint usually foresees:
the arrangement of the spatula 50 in the clamp consisting of the screw 23 and the projections 26 of the terminal 24;
the rotation of the screw 23 about the axis aa until the spatula 50 is rendered immobile in its position;
the locking through stops of the screw 23.

FIG. 16 also illustrates the joint 5 clamping the load cell 30a. So that it can measure the force exerted by the locking screw 23 on the spatula 50 to keep it firmly attached to the terminal 24, the load cell 30a is arranged inside the clamp consisting of the screw 23 and the projections 26 of the terminal 24 so as to lie down on the surface of the spatula 50 subjected to locking.

The present invention therefore also refers to a detection system 40 in which the measurements obtained by the sensors of the retractor system are acquired through an electronic measuring system interfaced with a microprocessor calculation device (or computer), in order to:
make the signals corresponding to the measurements discrete, sampling them, quantifing them and digitising them;
store said signals on magnetic and/or optical mass storage supports, which can be fixed or removable (of the “plug & play” type);
possibly, display said signals by means of a suitable peripheral video device (“monitor”).

In other words, the detection system according to the invention comprises a retractor system and an external device for collecting and storing the characteristic parameters of the organic tissues at a point of contact with the spatula body 1, as well as at least one electrical cable 8 coated with an insulating sheath for the connection to a sensor housed in the spatula body 1.

Advantageously, it is also foreseen that there be an electronic device (or “keyboard”) for the user to interact with the computer so that the former can give display and/or storage commands of the acquired signals to the latter.

It should be noted that the interconnection between the sensors foreseen in each of the embodiments exemplified above and the electrical power supply, or else between these and the measurer, or else between the latter and the computer, or between the computer, the keyboard, the monitor and possible removable external mass storage systems, for safety reasons, is meant to be carried out through electrical wires coated with an insulating sheath that is sterile or able to be sterilised according to the procedures foreseen by the current sanitary standards of the national territory with regard to hygiene of surgical tools and apparatuses.

Furthermore, it should be noted that in all of the embodiments exemplified above the coating sheaths and the sensors integrated in them are meant to be single-use and sterile, as foreseen by the aforementioned standards. The spatulas, the articulated mechanical support systems, the terminals and the possible force sensors arranged on them, the measurer, the computer, the monitor, the keyboard and possible removable external mass storage supports, on the other hand, are meant to be treatable and/or sterilisable according to the procedures foreseen by the aforementioned standards.

Basically, the invention provides a sensorised surgical device for multi-parameter monitoring in the form of a retractor system for surgical applications equipped with at least one sensor integrated with it.

Such a retractor system comprises at least one spatula for surgical applications, in particular coated with a sheath of bio-compatible material. Advantageously, according to the invention, the system comprises at least one from: a sensor for force measurements, a sensor for measuring the oximetry of the blood, a temperature sensor and a sensor for detecting and registering the electrical neurone activity (or “electrode”).

It should be noted that the presence of such an electrode is advisable in particular in the case in which the retractor system is intended for uses in the field of neurosurgical interventions.

Advantageously, according to the invention, the proposed retractor system allows at least one from:

the measurement of the pressure exerted by the spatula on the organic tissue (for example, brain tissue) with which it interacts;
the measurement of the degree of oximetry reached by the organic tissue at the point of contact with the spatula;
the measurement of the temperature of the portion of organic tissue in contact with the spatula;
the measurement of the electrical neurone activity, in the case of application of the spatula to brain tissue.

In this way, the retractor system is particularly advantageous in the field of neurosurgery as an instrument for helping the surgeon capable of reducing the risks connected with the effect of the pressure on the brain tissue exerted by the spatula during a surgical intervention. The combined knowledge of the pressure and oximetry measurements, indeed, together with the evaluation of the level of electrical neurone activity, allows the maximum pressure that can be exerted with the spatulas without the consequent reduction in oximetry producing necrosis of the neurones with damage to the patient to be established, in relation to the type and length of the intervention, allowing the surgeon to modify how the surgery is performed during surgery itself.

Advantageously, according to the invention, the proposed retractor system increases the safety of the patient when undergoing surgery.

Finally, it should be noted that what has been provided in the present description constitutes exclusively a limited group of specific embodiments of the invention supplied for illustrative purposes. Various modifications can be made to them without departing from the spirit and the purposes of the same invention. Therefore, said invention is not limited save for the claims shown hereafter.

1-37. (canceled)

38. A retractor system for retracting organic tissues in an area where a surgical operation is to be performed, the retractor system comprising:
at least one spatula comprising a spatula body, and a bio-compatible material layer thereon; and
at least one sensor carried by said at least one spatula and being at least partially coated with said bio-compatible material layer to detect at least one organic tissue characteristic parameter.

39. The retractor system according to claim 38, wherein said spatula body has at least one cavity therein receiving said at least one sensor; and further comprising an insulating adhesive material layer coupling said at least one sensor to said spatula body.

40. The retractor system according to claim 38, further comprising at least one electrical wire in said bio-compatible material layer for electrical connection to said at least one sensor.

41. The retractor system according to claim 38, wherein said spatula body comprises at least one conductive pathway therein for leading an electrical connection to said at least one sensor.

42. The retractor system according to claim 39, wherein said at least one sensor comprises a force measurer to measure a force applied by said spatula body to the organic tissues.

43. The retractor system according to claim 42, wherein said force measurer comprises an extensometer, and at least one spring projecting from said spatula body and being sterile and comprising a bio-compatible material; and wherein said at least one spring is positioned to at least partially cover said cavity at a proximal portion thereof at a surface of said spatula body and coupled to said extensometer.

44. The retractor system according to claim 42, wherein said force measurer comprises an extensometer system positioned to at least partially cover said cavity at a proximal portion thereof at a surface of said spatula body.

45. The retractor system according to claim 44, wherein said extensometer system comprises a plurality of extensometers including a single membrane of at least one of a conductive and piezoresistive material; and wherein the plurality of extensometers include no overlap between corresponding useful measurement surfaces.

46. The retractor system according to claim 38, wherein said at least one sensor comprises a force measurer comprising a load cell for measuring a force applied by said spatula body to the organic tissues.

47. The retractor system according to claim 38, wherein said at least one sensor comprises a blood oximetry measurer for measuring the degree of oximetry reached by the organic tissues.

48. The retractor system according to claim 47, wherein said blood oximetry measurer comprises at least one infrared emitter and an infrared receiver and having at least one useful measurement surface along a work surface of said spatula body.

49. The retractor system according to claim 48, wherein said at least one infrared emitter and said infrared receiver do not project from the work surface of said spatula body and are at least partially exposed by said bio-compatible material layer.

50. The retractor system according to claim 38, wherein said at least one sensor comprises a conductive material electrode for detecting electrical neurone activity of the organic tissues.

51. The retractor system according to claim 38, wherein said at least one sensor comprises a temperature measurer for measuring the temperature of the organic tissues.

52. The retractor system according to claim 51, wherein said temperature measurer comprises a thermistor.

53. The retractor system according to claim 38, wherein said bio-compatible material layer comprises at least one of silicon and a compound thereof.

54. The retractor system according to claim 38, wherein said at least one spatula further comprises a joint and a suspension system for coupling to said joint at a point of said spatula body not coinciding with a point of contact with the organic tissues.

55. The retractor system according to claim 54, wherein said joint comprises a force measurer.

56. The retractor system according to claim 55, wherein said force measurer comprises a load cell.

57. The retractor system according to claim 56, wherein said joint comprises a rigid structure for connection with said spatula body; wherein said rigid structure comprises a projection for housing said force measurer; wherein said joint comprises a mobile element coupled to said rigid structure, said mobile element being moveable in abutment on said force measurer for holding said force measurer.

58. The retractor system according to claim 38, wherein said at least one sensor comprises a plurality of sensors each having different characteristic parameters of the organic tissues, said plurality of sensors each having respective useful measuring surfaces not overlapping each other.

59. The retractor system according to claim 38, wherein said at least one spatula comprises a plurality of spatulas.

60. The retractor system according to claim 38, wherein the retractor system is sterile and single-use.

61. The retractor system according to claim 38, wherein the retractor system is re-useable.

62. The retractor system according to claim 38, wherein said spatula also comprises a sheath for covering said spatula body; and wherein said least one sensor and at least one conductive pathway are integrated in said sheath.

63. The retractor system according to claim 38, wherein said at least one sensor comprises at least one fiber optic sensor.

64. A retractor system for retracting organic tissues in an area where a surgical operation is to be performed, the retractor system comprising:
at least one spatula comprising a spatula body, and a bio-compatible material layer thereon; and
at least one sensor carried by said at least one spatula and being at least partially coated with said bio-compatible material layer to detect at least one organic tissue characteristic parameter;
said at least one sensor comprising at least one spring projecting from said spatula body and being for measuring a force applied by said spatula body on the organic tissues at a contact point therewith.

65. The retractor system according to claim 64, wherein said spatula body has at least one cavity therein receiving said at least one sensor; and further comprising an insulating adhesive material layer coupling said at least one sensor to said spatula body.

66. The retractor system according to claim 64, wherein said spatula body comprises at least one conductive pathway therein for leading an electrical connection to said at least one sensor.

67. The retractor system according to claim 64 wherein said at least one sensor is elastically deformable in contact with the organic tissues.

68. A detection system comprising:
a retractor system for retracting organic tissues in an area where a surgical operation is to be performed, the retractor system comprising
at least one spatula comprising a spatula body, and a bio-compatible material layer thereon, and
at least one sensor carried by said at least one spatula and at least partially coated with said bio-compatible material layer to detect at least one organic tissue characteristic parameter; and
an external device for collecting and storing the at least one organic tissue characteristic parameter.

69. The detection system according to claim 68 wherein said external device comprises at least one electrical cable for connection to said at least one sensor.

70. The detection system according to claim 68 further comprising a power supply device coupled to said at least one sensor.

71. The detection system according to claim 70 wherein said power supply device is integrated in said external device.

72. The detection system according to claim 68 wherein said external device comprises an electronic measuring system for acquiring signals generated downstream of the measurement being carried out for at least one of conditioning, digitizing, and numerically filtering the signals.

73. The detection system according to claim 72 wherein said electronic measuring system comprises at least one A/D (analog/digital) interface and software for acquiring, sampling, and quantifying the signals.

74. The detection system according to claim 73 wherein said external device also comprises a microprocessor calculation device for acquiring the signals for at least one of processing, displaying and storing the signals.

75. The detection system according to claim 74 wherein said external device further comprises an electronic keyboard device coupled to said microprocessor calculation device through which a user inserts storage and display commands.

76. The detection system according to claim 75 wherein said external device comprises an electronic display device for displaying data acquired and processed by said microprocessor calculation device coupled thereto.

77. A method of making a retractor system for retracting organic tissues in an area where a surgical operation is to be performed, the method comprising:
forming at least one spatula comprising a spatula body and a bio-compatible material layer thereon; and
coupling at least one sensor with the at least one spatula so that the at least one sensor is at least partially coated with the bio-compatible material layer to detect at least one organic tissue characteristic parameter.

78. The method according to claim 77, wherein forming at least one spatula body comprises forming at least one cavity therein for receiving the at least one sensor; and further comprising forming an insulating adhesive material layer coupling the at least one sensor to the at least one spatula body.

79. The method according to claim 77, further comprising forming at least one electrical wire in the bio-compatible material layer for electrical connection to the at least one sensor.

80. The method according to claim 78, wherein forming at least one spatula body comprises forming at least one conductive pathway therein for leading an electrical connection to the at least one sensor.
